(19) United States
(12) Patent Application Publication
(10) Pub. No.: US 2003/0175334 A1
(43) Pub. Date: Sep. 18, 2003

(54) Phospholipid bodies and use thereof in medical treatment

(76) Inventors: Anthony E. Bolton, Derbyshire (GB); Arkady Mandel, North York (CA)

Correspondence Address: Gerald F. Swiss, Esq., BURNS, DOANE, SWECKER & MATHIS, L.L.P, P.O. Box 1404, Alexandria, VA, 22313-1404, US

(21) Appl. No.: 10/348,600

(22) Filed: Jan. 21, 2003

Related U.S. Application Data
(60) Provisional application No. 60/351,427, filed on Jan. 28, 2002.
(60) Provisional application No. 60/364,620, filed on Mar. 18, 2002.
(60) Provisional application No. 60/372,106, filed on Apr. 15, 2002.
(60) Provisional application No. 60/400,857, filed on Aug. 2, 2002.

(30) Foreign Application Priority Data
Jan. 21, 2002 (CA) 2,368,656

(51) Int. Cl. A61K 31/685; A61K 9/127

(52) U.S. Cl. 424/450; 514/78

(57) ABSTRACT

This invention relates to synthetic and semi-synthetic compositions having biological activity, and to the uses thereof in the treatment and/or prophylaxis of various disorders in mammalian patients. More particularly it relates to preparation and use of synthetic and semi-synthetic bodies, which after introduction into the body of a patient, produce beneficial anti-inflammatory, organ protective and immune regulatory effects. The invention also relates to treatments and compositions for alleviating inflammatory and autoimmune diseases and their symptoms.

### CROSS-REFERENCE TO RELATED APPLICATIONS

This application claims the benefit under 35 U.S.C. § 119(a) of Canadian Application No. 2,368,656, filed on Jan. 21, 2002, which application is herein incorporated by reference in its entirety.

This application further claims the benefit under 35 U.S.C. § 119(e) of the following applications: U.S. Provisional Application No. 60/___,___, which was converted pursuant to 37 C.F.R. § 1.53(c)(2)(i) from U.S. patent application Ser. No. 10/051,381, filed Jan. 22, 2002; U.S. Provisional Application No. 60/351,427, filed Jan. 28, 2002; U.S. Provisional Application No. 60/364,620, filed Mar. 18, 2002; U.S. Provisional Application 60/372,106, filed Apr. 15, 2002 and U.S. Provisional Application No. 60/400,857, filed Aug. 2, 2002, all of which applications are herein incorporated by reference in their entireties.

### BACKGROUND OF THE INVENTION

1. Field of the Invention

This invention relates to synthetic and semi-synthetic compositions having biological activity, and to the uses thereof in the treatment and/or prophylaxis of various disorders in mammalian patients. More particularly it relates to preparation and use of synthetic and semi-synthetic bodies, which after introduction into the body of a patient, produce beneficial anti-inflammatory, organ protective and immune regulatory effects. The invention also relates to treatments and compositions for alleviating inflammatory and autoimmune diseases and their symptoms.

2. Background of the Invention

Professional antigen-presenting cells (APCs), including dendritic cells (DCs) and macrophages (Mph), are cell types that actively capture and process antigens (Ags) and remove infectious organisms, cell debris and dying cells including the residues from dying cells. During this process, APCs can stimulate the production of either pro-inflammatory Thp1 cytokines (IL-12, IL-1, INF-γ, TNF-α, etc.) or regulatory/anti-inflammatory Th2/Th3 cytokines (such as IL-10, TGF-γ, IL-4 etc.) dominated responses; depending on the nature of the Ag or cell residues encountered and the level of APC maturation/activation.

APCs remove cellular debris, some of which is derived from cell membranes of the body, some from bacterial and parasitic infections and commensal organisms such as gut bacteria. While some of this cellular debris will initiate a pro-inflammatory response some will initiate a protective and anti-inflammatory response.

A normally functioning immune system is capable of distinguishing between the antigens of foreign invading organisms (non-self) and tissues or debris derived from “self”, mounting an immune response only against foreign antigens. When a patient's immune system fails to discriminate between self and non-self, autoimmune disorders arise.

3. References

1. U.S. Pat. No. 4,485,054, issued Nov. 27, 1984, to Mezei et al.

2. U.S. Pat. No. 4,496,787, issued Jan. 29, 1985, to Touchais et al.

3. U.S. Pat. No. 4,812,314, issued Mar. 14, 1989 to Barenholz.

4. U.S. Pat. No. 4,938,763, issued Jul. 3, 1990 to Dunn et al.

5. U.S. Pat. No. 4,946,787, issued Aug. 7, 1990, to Eppstein et al.

6. U.S. Pat. No. 5,188,951, issued Feb. 23, 1993, to Tremblay et al.

7. U.S. Pat. No. 5,252,263, issued Oct. 12, 1993, to Hope et al.

8. U.S. Pat. No. 5,376,452, issued Dec. 27, 1994, to Hope et al.

9. U.S. Pat. No. 5,736,157, issued Apr. 7, 1998, to Williams.

10. U.S. Pat. No. 5,741,514, issued Apr. 21, 1998, to Barenholz et al.

11. U.S. Pat. No. 5,746,223, issued May 5, 1998, to Williams.

12. U.S. Pat. No. 5,843,474, issued Dec. 1, 1998, to Williams.

13. U.S. Pat. No. 5,858,400, issued Jan. 12, 1999, to Williams.

14. U.S. Pat. No. 6,297,870, issued Oct. 2, 2001, to Nanba.

15. U.S. Pat. No. 6,312,719, issued Nov. 6, 2001, to Hope et al.

16. International Publication No. WO 01/66785, published Sep. 13, 2001.

17. International Patent Application PCT/CA02/01398 to Vasogen Ireland Limited.

18. Lehniger, Biochemistry (1970)

19. Barenholz et al. “Liposomes as Pharmaceutical Dosage Forms”

20. New, R. C. “Liposomes: A Practical Approach”, IRL Press at Oxford University Press (1990).

21. R. Harrigan—1992 University of British Columbia PhD Thesis “Transmembrane pH gradients in liposomes (microform): drug-vesicle interactions and proton flux”, published by National Library of Canada, (1993); University Microfilms order no. UM100406756; Canadian no. 942042220, ISBN 0315796936.

22. Griffin WST et al. “Brain interleukin 1 and S-100 immunoreactivity are elevated in Down Syndrome and Alzheimer Disease.” Proceedings of the National Academy of Sciences USA. 86: 7611-7615 (1989).

23. Bliss, T. V. P., et al. “A synaptic model of memory: long-term potentiation in the hippocampus.” Nature. 361: 31-39 (1993).

24. Murray, C. A., et al. “Evidence that increase hippocampal expression of the cytokine interleukin-1B is a common trigger for age and stress-induced impairments in long-term potentiation.” J. Neuroscience. 18: 2974-2981 (1998).

25. Mogi, M., et al. “Interleukin (IL)-1 beta, IL-1, IL-4, IL-6 and transforming growth factor-alpha levels are elevated in ventricular cerebrospinal fluid in juvenile parkinsonism and Parkinson's Disease.” Neuroscience Letters. 211: 13-16 (1996).

26. Giannessi D, Del Ry S, Vitale R L “The role of endothelins and their receptors in heart failure.” Pharmacol Res 2001 Feb 43:2 111-26.

27. Van de Stolpe A, Van der Saag P T, “Intercellular adhesion molecule-1” J. Mol. Med. (1996) 74:1 12-33.

All of the above publications, patents and patent applications are herein incorporated by reference in their entirety to the same extent as is if each individual publication, patent or patent application was specifically and individually indicated to be incorporated by reference in its entirety.

### SUMMARY OF THE INVENTION

This invention is directed to the discovery that bodies, such as liposomes, beads or similar particles, which comprise certain reactive chemical groups such as anionic phospholipid groups, other than phosphate-serine and phosphate-glycerol, will, upon administration to a mammalian patient, cause an anti-inflammatory effect and, therefore, may be used to treat a number of diseases. These bodies may also further comprise as a minor component on their surface an inactive constituent, and/or a constituent, which is active through a different mechanism.

In a preferred embodiment, the invention is directed to a composition of matter capable of producing an anti-inflammatory response in vivo in a mammal, said composition comprising pharmaceutically acceptable bodies of size from about 20 nanometers (nm) to 500 micrometers (μm), comprising a plurality of reactive chemical groups (hereinafter “anti-inflammatory promoting groups”) other than phosphate-serine and phosphate-glycerol, which interact with receptors on cells of the mammalian immune system to alter the cytokine profile of the immune system in favor of anti-inflammation. The anti-inflammatory promoting groups include active groups of certain anionic and other phospholipids, other than phosphate-serine and phosphate-glycerol, certain peptides and synthetic mimetics thereof, adaptor proteins, lipids, lipoproteins and the like, more specifically described below. The bodies, through the groups, are believed to interact with the immune system of a mammal after administration thereto, to produce an anti-inflammatory response in vivo in said mammal. Preferably, the bodies are essentially free of non-lipid pharmaceutically active entities. Preferably the groups constitute 60%-100% of the active surface groups on the bodies. The bodies may also comprise as a minor component on their surface an inactive constituent (such as phosphate-choline) in some indications and/or a constituent, which is active through another mechanism such as phosphate-serine.

In another embodiment, this invention is directed to a three-dimensional liposomes having been modified to comprise, as a major component, at least one anti- inflammatory promoting ligand wherein the ligand is an anionic phospholipid other than PG or PS, wherein PG and PS are as defined below.

In still another embodiment, this invention is directed to three-dimensional synthetic, semi-synthetic or natural bodies, otherwise referred to herein as pharmaceutically acceptable bodies, having sizes ranging from 20 nm to 500 micrometers, and having anti-inflammatory promoting groups other than phosphate-glycerol and phosphate-serine, on the surface thereof.

In another aspect, the invention is directed to a method for treating a T-cell function-mediated disorder comprising administering to a mammalian patient an effective amount of pharmaceutically acceptable bodies comprising an effective number of anti-inflammatory promoting groups as described herein, to inhibit and/or reduce the progression of the T-cell function-mediated disorder.

This invention is further directed to a method for treating an inflammatory disorder comprising administering to a patient an effective amount of pharmaceutically acceptable bodies comprising an effective number of anti-inflammatory promoting groups as described herein, to inhibit and/or reduce the progression of the inflammatory disorder.

Yet another embodiment of this invention is a method for treating an endothelial function disorder comprising administering to a mammalian patient an effective amount of pharmaceutically acceptable bodies comprising an effective number of anti-inflammatory promoting groups as described herein, to inhibit and/or reduce the progression of the endothelial function disorder.

Another embodiment is a method for treating an immune disorder characterized by inappropriate cytokine expression comprising administering to a mammalian patient an effective amount of pharmaceutically acceptable bodies comprising an effective number of anti-inflammatory promoting groups as described herein, to inhibit and/or reduce the progression of the immune disorder.

The invention is further directed to a method for treating or prophylaxis of a mammalian cardiac disorder, the presence of or the susceptibility to which is detectable by observing a prolonged QT-c interval on an electrocardiogram of the patient, which method comprises administering to the patient suffering therefrom or susceptible thereto a pharmaceutical composition comprising pharmaceutically acceptable biocompatible synthetic, semi-synthetic or natural bodies, otherwise referred herein as pharmaceutically acceptable bodies, and a pharmaceutically acceptable carrier, wherein at least a portion of said bodies are in the range from about 20 nm to 500 μm, and wherein the surfaces of said bodies have been modified to comprise, as a major component, at least one anti-inflammatory promoting group as described herein, said group being an anti-inflammatory group other than phosphate-glycerol and phosphate-serine.

Another embodiment of the invention is a pharmaceutical composition in unit dosage form, for administration to a mammalian patient, comprising pharmaceutically acceptable bodies and a pharmaceutically acceptable carrier, wherein at least a portion of the bodies have a size in the range from about 20 nm to 500 μm, and wherein the surfaces of said bodies comprise anti-inflammatory promoting groups, said unit dosage comprising from 500 to 2.5×10⁹ bodies, wherein said group is an anti-inflammatory promoting group, as described herein, other than phosphate-glycerol and phosphate-serine.

A further embodiment of this invention is a pharmaceutical composition comprising pharmaceutically acceptable biocompatible synthetic, semi-synthetic or natural bodies (otherwise referred to herein as pharmaceutically acceptable bodies) and a pharmaceutically acceptable carrier, wherein at least a portion of said bodies has a size from about 20 nm to 500 μm, and wherein the surfaces of said bodies have been modified to comprise, as a major component, at least one anti-inflammatory promoting group, wherein said group is an anti-inflammatory promoting group, as described herein, other than phosphate-glycerol and phosphate-serine.

Optionally the bodies described above may in addition comprise an inactive constituent surface group and/or a constituent surface group that is active through another mechanism, e.g. phosphatidylserine (e.g. see Fadok et al., International Publication WO 01/66785).

In another embodiment, this invention is directed to lyophilized or freeze-dried pharmaceutically acceptable bodies comprising anti-inflammatory promoting ligand groups as binding groups, and kits comprising lyophilized or freeze dried bodies comprising anti-inflammatory promoting groups or groups convertible to anti-inflammatory promoting groups, and a pharmaceutically acceptable carrier, wherein said group is an anti-inflammatory promoting group as described herein, other than phosphate-glycerol and phosphate-serine.

In another aspect, this invention is directed to a method for treating a T-cell function-mediated disorder comprising administering to a mammalian patient suffering from or at risk of suffering from a T-cell function-mediated disorder, an effective amount of a composition comprising pharmaceutically acceptable bodies having a size from about 20 nm to about 500 μm, comprising on the surface thereof a plurality of anti-inflammatory promoting groups other than phosphate-glycerol or phosphate-serine, or groups convertible to said anti-inflammatory promoting groups, as described herein such that upon administration, the progression of the T-cell function mediated disorder is inhibited and/or reduced.

Yet another embodiment of this invention is directed to a method for treating an endothelial function disorder comprising administering to a mammalian patient suffering from or at risk of suffering from an endothelial function disorder an effective amount of a composition comprising pharmaceutically acceptable bodies having a size from about 20 nm to about 500 μm, comprising on the surface thereof a plurality of anti-inflammatory promoting groups other than phosphate-glycerol or phosphate-serine, or groups convertible to said anti-inflammatory promoting groups, as described herein, such that upon administration, the progression of the endothelial function mediated disorder is inhibited and/or reduced.

Another embodiment of this invention is directed to a method for treating an immune disorder comprising administering to a mammalian patient suffering from or at risk of suffering from an immune disorder an effective amount of a composition comprising pharmaceutically acceptable bodies having a size from about 20 nm to about 500 μm, comprising on the surface thereof a plurality of anti-inflammatory promoting groups other than phosphate-glycerol or phosphate-serine, or groups convertible to said anti-inflammatory promoting groups, as described herein, such that upon administration, the progression of the immune disorder is inhibited and/or reduced.

Another embodiment of this invention is directed to a method for treating an inflammatory disorder comprising administering to a mammalian patient suffering from or at risk of suffering from an inflammatory disorder an effective amount of a composition comprising pharmaceutically acceptable bodies having a size from about 20 nm to about 500 μm, comprising on the surface thereof a plurality of anti-inflammatory promoting groups, as described herein, other than phosphate-glycerol or phosphate-serine, or groups convertible to said anti-inflammatory promoting groups, such that upon administration, the progression of the inflammatory disorder is inhibited and/or reduced.

### BRIEF DESCRIPTION OF THE DRAWINGS

FIG. 1 of the accompanying drawings is a bar graph presentation of the results obtained in Example 1 below.

FIG. 2 is a bar graph presentation of the results obtained according to Example 2 below.

FIG. 3 similarly presents the results obtained according to Example 3 below.

FIG. 4 similarly presents the results obtained according to Example 4 below.

FIG. 5 similarly presents the results obtained according to Example 5 below.

### DESCRIPTION OF PREFERRED EMBODIMENTS

According to the present invention, pharmaceutically acceptable bodies comprising anti-inflammatory promoting ligand groups on their surface are administered to patients. Without being limited to any theory, it is believed that these bodies interact with the immune system of the patient with accompanying beneficial effects such as inhibition of pro-inflammatory cytokines in vivo and/or promotion of anti-inflammatory cytokines. The reacting cells may be immune cells such as professional or non-professional antigen presenting cells, endothelial cells, regulatory cells such as NK-T cells and others.

These pharmaceutically acceptable bodies include synthetic, semi-synthetic and natural bodies having shapes which are typically but not exclusively spheroidal, cylindrical, ellipsoidal, including oblate and prolate spheroidal, serpentine, reniform etc., and sizes from about 20 nm to about 500 μm in diameter, preferably measured along its longest axis.

The pharmaceutically acceptable bodies have one or more anti-inflammatory promoting groups of predetermined characteristics on the exterior surface in a manner, in one embodiment, that they are capable of interacting with the appropriate receptor(s), other than exclusively the PS receptor or the PG receptor, on antigen presenting cells in vivo. The structure of these groups may be synthetically altered and include all, part of or a modified version of the original anti-inflammatory promoting group.

As used herein the term “PG” is intended to cover phospholipids comprising a phosphate-glycerol group with a wide range of at least one fatty acid chains provided that the resulting PG entity can participate as a structural component of a liposome. Preferably, such PG compounds can be represented by the Formula I:

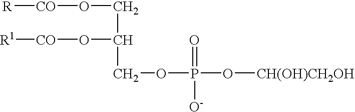

where R and R¹ are independently selected from C₁-C₂₄ hydrocarbon chains, saturated or unsaturated, straight chain or containing a limited amount of branching wherein at least one chain has from 10 to 24 carbon atoms. Essentially, the lipid chains R and R¹ form the structural component of the liposomes, rather than the active component. Accordingly, these can be varied to include two or one such lipid chains, the same or different, provided they fulfill the structural function. Preferably, the lipid chains may be from about 10 to about 24 carbon atoms in length, saturated, mono-unsaturated or polyunsaturated, straight-chain or with a limited amount of branching. Laurate (C12), myristate (C14), palmitate (C16), stearate (C18), arachidate (C20), behenate (C22) and lignocerate (C24) are examples of useful saturated lipid chains for the PG for use in the present invention. Palmitoleate (C16), oleate (C18) are examples of suitable mono-unsaturated lipid chains. Linoleate (C18), linolenate (C18) and arichidonate (C20) are examples of suitable poly-unsaturated lipid chains for use in PG in the liposomes of the present invention. Phospholipids with a single such lipid chain, also useful in the present invention, are known as lysophospholipids. The present invention also extends to cover use of liposomes in which the active component is the dimeric form of PG, namely cardiolipin but other dimers of Formula I are also suitable. Preferably, such dimers are not synthetically cross-linked with a synthetic cross-linking agent, such as maleimide but rather are cross-linked by removal of a glycerol unit as described by Lehniger, Biochemistry, p. 525 (1970) and depicted in the reaction below:

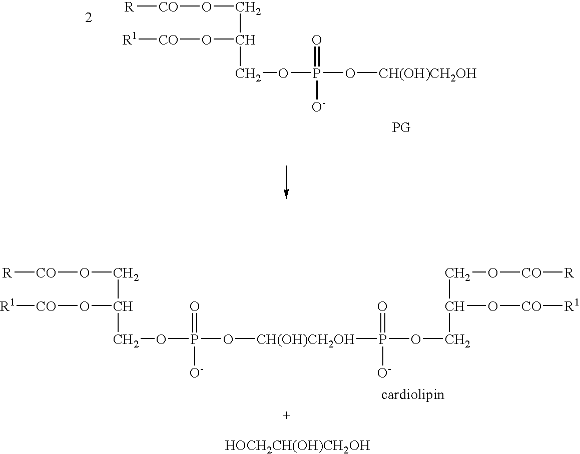

where each R and R¹ are independently as defined above.

As used herein the term “PS” is intended to cover phosphatidylserine and analogues/derivatives thereof provided that such analogues/derivatives enhance or stimulate the activity of the phosphatidylserine receptor.

When the pharmaceutically acceptable bodies used herein are liposomes derived from one or more phospholipids such lipids preferably do not comprise PS or PG.

In a preferred embodiment these anti-inflammatory promoting groups are anionic phospholipids other than phosphate-glycerol or phosphate-serine. In a most preferred embodiment the anionic phospholipid is phosphatidylinositol. Inositol, hexahydroxycyclohexane, chemically links to the phosphate group in phosphatidylinositol PI through one of its hydroxyl groups. There are a variety of stereoisomers of inositol, relating to the disposition of the hydoxyl groups relative to the nucleus. All such stereoisomeric forms of PI are embraced within the terms phosphatidylinositol and PI as used herein.

Analogues of phosphatidylinositol with modified active groups, which also interact with PI receptors on the antigen presenting cells, or otherwise result in an anti-inflammatory reaction in the recipient body are contemplated within the scope of the term phosphatidylinositol. This includes, without limitation, compounds in which one or more of the hydroxyl groups and/or the phosphate group is derivatized, or in the form of a salt. Many such compounds form free hydroxyl groups in vivo, upon or subsequent to administration.

Naturally occurring phosphatidylinositol (PI) is a minor natural phospholipid in cell membranes. Chemically, it has a phospho-inositol group, a glycerol group and a pair of similar but different C₁₈-C₂₀ fatty acid chains, so that it can be represented by the chemical formula II:

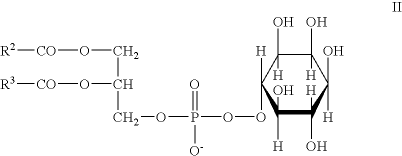

where R² represents arichidonate (C₂₀, Δ5, 8,11,14) and R³ represents stearate (C₁₈, saturated). While the natural PI compound as above constitutes the most preferred active constituent of the liposomes used in the present invention, variations in the nature and number of the lipid chains are within the scope. Essentially, the lipid chains form the structural component of the liposomes, rather than the active component. Accordingly, these can be varied to include two or one such lipid chains, the same or different, provided they fulfill the structural function. Preferably, the lipid chains may be from about 10 to about 24 carbon atoms in length, saturated, mono-unsaturated or polyunsaturated, straight-chain or with a limited amount of branching. Laurate (C12), myristate (C14), palmitate (C16), stearate (C18), arachidate (C20), behenate (C22) and lignocerate (C24) are examples of useful saturated lipid chains for the PI for use in the present invention. Palmitoleate (C16), oleate (C18) are examples of suitable mono-unsaturated lipid chains. Linoleate (C18), linolenate (C18) and arichidonate (C20) are examples of suitable poly-unsaturated lipid chains for use in PI in the liposomes of the present invention. The stereo configuration of the lipid chain is unimportant, and all such stereo isomers are embraced in the present definition. Phospholipids with a single such lipid chain, also useful in the present invention, are known as lysophospholipids. All such variations are included within the definitions of PI.

As used herein, the term “phosphate-inositol group” refers to the following formula:

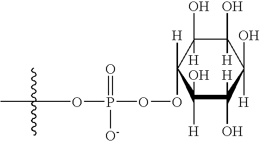

which is sometimes referred to as “ligand,” “head group,” “active group,” or “binding group.”

The present invention can also be viewed, from another aspect, as the use of a receptor on cells of the mammalian immune system, e.g. macrophages, which specifically bind to the phosphate-inositol group. The invention embraces bodies comprising ligands and groups that will bind to such receptor and consequently produce an anti-inflammatory response. Accordingly, the present invention can be defined as bodies comprising ligands or active groups thereof that compete with the binding or uptake of phosphate-inositol expressing bodies as described herein by antigen-presenting cells. A person skilled in the art can readily determine whether a particular body is one, which will so compete, by conducting simple test experiments. For example, the bodies can be tested with a readily available monocytic cell line such as U937 cells. In a first experiment, U937 cells are incubated with fluorescently labeled PI liposomes alone, and in other experiments the U937 cells are incubated in the presence of both fluorescently labeled PI liposomes and differing amounts of test compound. If the uptake of the fluorescently labeled PI liposomes in the other experiments is reduced in comparison with that of the first experiment, then the test compound is competing for the specific receptor and is a compound within the scope of the present invention.

It is also within the scope of the present invention to use bodies having a mixture of the aforementioned phospholipids having chemically active groups, this mixture comprising at least 10%, preferably at least 50% and most preferably 60-90% of the aforementioned active phospholipids such as PI. Instead of the minor constituent being an inactive constituent, it can be active through another mechanism.

Examples of “three-dimensional body portions” or “pharmaceutically acceptable bodies” include biocompatible synthetic, semi-synthetic or natural entities such as liposomes, solid beads, hollow beads, filled beads, particles, granules and microspheres of biocompatible materials, natural or synthetic, such as polyethylene glycol, polyvinylpyrrolidone, polystyrene, poly(methylmethacrylate), etc., polysaccharides such as hydroxyethyl starch, hydroxyethylcellulose, agarose and the like, as commonly used in the pharmaceutical industry. The beads may be solid or hollow, or filled with biocompatible material. The term “biocompatible” refers to substances, which in the amount employed are neither non-toxic or have acceptable toxicity profiles such that their use in vivo is acceptable. Such bodies can include liposomes formed of lipid, one of which is phosphatidylinositol (PI) for example. Alternatively, the pharmaceutically acceptable bodies can be solid beads, hollow beads, filled beads, particles, granules and microspheres of biocompatible materials which comprise one or more biocompatible materials such as polyethylene glycol, poly(methylmethacrylate), polyvinylpyrrolidone, polystyrene and a wide range of other natural, semi-synthetic and synthetic materials, with anti-inflammatory promoting groups other than phosphate-glycerol or phosphate serine attached thereto.

As noted above, analogues of phosphatidylinositol with modified active groups, which also interact with the PI receptors on antigen presenting cells, through the same receptor pathway as PI or otherwise resulting in an anti-inflammatory reaction in the recipient body are contemplated within the scope of the term phosphatidylinositol. This includes, without limitation, compounds in which one or more of the hydroxyl groups and/or the phosphate group is derivatized, or in the form of a salt. Many such compounds form free hydroxyl groups in vivo, upon or subsequent to administration and, accordingly, comprise PI groups.

Preferred compositions of matter are liposomes, which may be composed of a variety of lipids. Preferably, none of the active lipids are positively charged. Liposomes, or lipid vesicles, are sealed sacs, in the micron or sub-micron range, the walls of which consist of suitable amphiphiles. They normally contain an aqueous medium. Generally the liposomes are composed of phosphatidylcholine (PC), distearoylphosphatidylcholine, phosphatidylinositol, phosphatidic acid, lysophosphatidic acid, lysophosphatidylinositol, lecithin, cephalin, cerebrosides including sphingomyelin, and sphingosine. Such liposomes are prepared and treated so that the active polar groups are presented exteriorly on the liposomal body. Thus a preferred embodiment of this invention provides synthetic or semi-synthetic or natural bodies, which expose or can be treated or induced to expose, on their surfaces the active anti-inflammatory promoting groups derived from one or more phospholipid ligands. These phospholipids will be found among phosphatidylcholine, phosphatidylethanolamine, phosphatidylinositol, phosphatidic acid, lysophosphatidic acid, lysophosphatidylinositol, lysophosphatidylcholine, lysophosphatidylethanolamine, sphingosylphosphoryl choline, sphingosine-1-phosphate, ceramides, sphingomyelin, and combinations of two or more thereof. Some of these are active, and others of these, e.g., phosphatidylcholine, are believed to be inactive in most applications but provide structure to the liposome.

Liposomes, or lipid vehicles, are scaled sacs, in the micron or sub-micron range, the walls (monolayer or multilayer) of which comprise suitable amphiphiles. They normally contain an aqueous medium, although for the present invention the interior contents are unimportant, and generally inactive. Accordingly, in a preferred embodiment, the liposomes, as well as other pharmaceutically acceptable bodies, are essentially free of non-lipid pharmaceutically acceptable entities (e.g. <1%) and more preferably are free of non-lipid pharmaceutically acceptable entities. Such liposomes are prepared and treated so that the active groups are presented exteriorly on the liposomal body. The phospholipids of the preferred embodiments of this invention thus serve as ligands and structural components of the liposome itself.

Thus, a preferred embodiment of this invention provides liposomal bodies which expose or can be treated to expose, on their surfaces, one or more groups, preferably phosphate-inositol, to act as binding groups. Non-PS/non-PG active phospholipids should comprise from 10%-100% of the liposome with the balance being an inactive constituent, e.g. phosphatidylcholine, or one that acts through a different mechanism e.g. phosphatidylserine, or mixtures of such. Inactive constituents such as PC are preferred.

At least 10% by weight of such liposome is composed of one or more of the non-PS/PG phospholipids having active anti-inflammatory promoting groups, preferably at least 50%, more preferably from 60-100% and most preferably from 70-90%, with the single most preferred embodiment being about 75% by weight of active phospholipid.

The preferred phospholipids for use in the present invention are phosphatidylinositol and phosphatidic acid, optionally with a minor portion (up to less than 50% by weight) of another, inactive constituent and/or a constituent active by another mechanism, i.e. phosphate-serine. The most preferred active phospholipid to be used in the present invention is phosphatidylinositol (PI), even more preferably a liposome constituted to the extent of about 70-90% PI, balance inactive phospholipid.

Mixtures of liposomes of different aforementioned phospholipids having chemically active groups, and mixtures of such liposomes with liposomes of inactive and/or with liposomes of phospholipids acting through a different mechanism can also be used, provided that the total amount of active phospholipid remains above the minimum of about 10% and preferably above 60% in the total mixture.

As regards to non-liposomal bodies for use in the present invention, these as noted include biocompatible solid or hollow beads of appropriate size. The biocompatible non-liposomal synthetic or semi-synthetic bodies may be selected from polyethylene glycol, poly(methylmethacrylate), polyvinylpyrrolidone, polystyrene and a wide range of other natural, semi-synthetic and synthetic materials. Such materials include biodegradable polymers, such as disclosed by Drum, et al. U.S. Pat. No. 4,938,763, which is hereby incorporated by reference in its entirety.

Biodegradable polymers are disclose in the art and include, for example, linear-chain polymers such as polyactides, polyglycolides, polycaprolactones, polyanhydrides, polyamides, polyurethanes, polyesteramides, polyorthoesters, polyioxanones, polyacetals, polyketals, polycarbonates, polyorthocarbonates, polyphosphazenes, polyhydroxybutyrates, polyhydroxyvalerates, polyalkylene, oxalates, polyalkylene succinates, poly(malic acid), poly(amino acids), polyvinylpyrrolidone, polyethylene glycol, polyhydroxycellulose, chitin, chitosan, and copolymers, terpolymers, and combinations thereof. Other biodegradable polymers include, for example, gelatin, collagen etc.

Suitable substances for derivatization to attach the phospholipid(s), or portions thereof with groups or binding groups, to three-dimensional bodies are commercially available e.g. from Polysciences Inc., 400 Valley Road, Warrington, Pa. 18976, or from Sigma Aldrich Fine Chemicals. Methods for their derivatization are known in the art. Specific preferred examples are disclosed in the International Patent Application PCT/CA02/01398 Vasogen Ireland Limited, which is incorporated herein by reference.

It is contemplated that the patient may be a mammal, including but not limited to humans and domestic animals such as cows, horses, pigs, dogs, cats and the like.

Phospholipids are amphiphilic molecules (i.e. amphiphiles), meaning that the compound comprises molecules having a polar water-soluble group attached to a water-insoluble hydrocarbon chain. The amphiphiles serving as the layers of the matrix have defined polar and apolar regions. The amphiphiles can include, in addition to the phospholipids providing the active groups in the process of the invention, other, naturally occurring lipids used alone with the phospholipid comprising the active group, or in a mixture with another. The amphiphiles serving as the layer(s) of the liposomes can be inert, structure-conferring synthetic compounds such as polyoxyethylene alkylethers, polyoxyethylene alkylesters and saccharosediesters.

Methods of preparing liposomes of the appropriate size are known in the art and do not form part of this invention. Reference may be made to various textbooks and literature articles on the subject, for example, the review article “Liposomes as Pharmaceutical Dosage Forms”, by Yechezkel Barenholz and Daan J. A. Chrommelin, and literature cited therein, for example New, R. C. “Liposomes: A Practical Approach”, IRL Press at Oxford University Press (1990).

The diameter of the liposomes as well as other pharmaceutically acceptable bodies, of the preferred embodiment of this invention is from about 20 nm to about 500 μm, more preferably from about 20 nm to about 1000 nm, more preferably from about 50 nm to about 500 nm, and most preferably from about 80 nm to about 120 nm (preferably measured along its longest axis).

The pharmaceutically acceptable bodies may be suspended in a pharmaceutically acceptable carrier, such as physiological sterile saline, sterile water, pyrogen-free water, isotonic sterile saline, and phosphate buffer sterile solutions, as well as other non-toxic compatible substances used in pharmaceutical formulations. Preferably, the pharmaceutically acceptable bodies are constituted into a liquid suspension in a biocompatible liquid such as buffered sterile saline and administered to the patient in any appropriate route which introduces it to the immune system, such as intra-arterially, intravenously or most preferably intramuscularly or subcutaneously.

It is contemplated that the pharmaceutically acceptable bodies may be freeze-dried or lyophilized so that they may be later resuspended for administration. This invention is also directed to a kit of parts comprising lyophilized or freeze-dried bodies comprising anti-inflammatory promoting groups, and a pharmaceutically acceptable carrier, such as physiological sterile saline, sterile water, pyrogen-free water, isotonic sterile saline, and phosphate buffer sterile solutions, as well as other non-toxic compatible substances used in pharmaceutical formulations.

A preferred manner of administering the pharmaceutically acceptable bodies to the patient is a course of injections, administered daily, several times per week, weekly or monthly to the patient, over a period ranging from a week to several months. The frequency and duration of the course of the administration is likely to vary from patient to patient, and according to the condition being treated, its severity, and whether the treatment is intended as prophylactic, therapeutic or curative. Its design and optimization is well within the skill of the attending physician. Intramuscular injection, especially via the gluteal muscle, is most preferred. One particular injection schedule, in at least some of the indication of the invention, is an injection, via the gluteal muscle, of an appropriate amount of bodies on day 1, a further injection on day 2, a further injection on day 14, and then “booster” injections at monthly intervals, if appropriate.

It is postulated that, in many embodiments of the present invention pharmaceutically acceptable bodies comprising anti-inflammatory promoting groups on their surface are acting as modifiers of the patient's immune system, in a manner similar to that of a vaccine. Accordingly they are used in quantities and by administration methods to provide a sufficient localized concentration of the bodies at the site of introduction. Quantities of such bodies appropriate for immune system modifying substances are generally not directly correlated with body size of a recipient and can, therefore, be clearly distinguished from drug dosages, which are designed to provide therapeutic levels of active substances in a patient's bloodstream and tissues. Drug dosages are accordingly likely to be much larger than immune system modifying dosages.

The correlation between weights of liposomes and numbers of liposomes is derivable from the knowledge, accepted by persons skilled in the art of liposomal formulations, that a 100 nm diameter bilayer vesicle has 81,230 lipid molecules per vesicle, distributed approximately 50:50 between the layers (see Harrigan—1992 University of British Columbia PhD Thesis “Transmembrane pH gradients in liposomes (microform): drug-vesicle interactions and proton flux,” published by National Library of Canada, Ottawa, Canada (1993); University Microfilms order no. UMI00406756; Canada no. 942042220, ISBN 0315796936). From this one can calculate, for example, that a dose of 5×10⁸ vesicles, of the order of the dose used in the specific in vivo examples below, is equivalent to 4.06×10¹³ lipid molecules. Using Avogadro's number for the number of molecules of lipid in a gram molecule (mole), 6.023×10²³, one determines that this represents 6.74×10⁻¹¹ moles which, at a molecular weight of 857 for PI, is approximately 5.78×10⁻⁸ g, or 57.8 ng of PI for such dosage.

The quantities of pharmaceutically acceptable bodies to be administered will vary depending on the nature of the mammalian disorder it is intended to treat and on the identity and characteristics of the patient. It is important that the effective amount of bodies is non-toxic to the patient, and is not so large as to overwhelm the immune system. When using intra-arterial, intravenous, subcutaneous or intramuscular administration of a liquid suspension of bodies, it is preferred to administer, for each dose, from about 0.1-50 ml of liquid, containing an amount of bodies generally equivalent to 10% - 1000% of the number of leukocytes normally found in an equivalent volume of whole blood. Generally, the number of bodies administered per delivery to a human patient is in the range from about 500 to about 2.5×10⁹ (<250 ng of bodies, in the case of liposomes, pro-rated for density differences for other embodiments of bodies), more preferably from about 1,000 to about 1,500,000,000, even more preferably 10,000 to about 100,000,000, and most preferably from about 200,000 to about 2,000,000.

Since the pharmaceutically acceptable bodies are acting, in the process of the invention, as immune system modifiers, in the nature of a vaccine, the number of such bodies administered to an injection site for each administration is a more meaningful quantitation than the number or weight of bodies per unit of patient body weight. For the same reason, it is now contemplated that effective amounts or numbers of bodies for small animal use may not directly translate into effective amounts for larger mammals (i.e. greater than 5 kg) on a weight ratio basis.

The present invention is indicated for use in prophylaxis and/or treatment of a wide variety of mammalian disorders where T-cell function, inflammation, endothelial dysfunction and inappropriate cytokine expression are involved. A patient having or suspected of having such a disorder may be selected for treatment. “Treatment” refers to a reduction of symptoms, such as, but not limited to, a decrease in the severity or number of symptoms of the particular disease or a limit on the further progression of symptoms.

With respect to T-cell function (T-cell mediated) disorders, these may be ulcers and wounds, and autoimmune disorders including, but not limited to diabetes, scleroderma, psoriasis and rheumatoid arthritis.

The invention is indicated for use with inflammatory allergic reactions, organ and cell transplantation reaction disorders, and microbial infections giving rise to inflammatory reactions. It is also indicated for use in prophylaxis against oxidative stress and/or ischemia reperfusion injury, ingestion of poisons, exposure to toxic chemicals, radiation damage, and exposure to airborne and water-borne irritant substances, etc., which cause damaging inflammation. It is also indicated for inflammatory, allergic and T-cell-mediated disorders of internal organs such as kidney, liver, heart, etc.

With respect to disorders involving inappropriate cytokine expression for which the present invention is indicated, these include neurodegenerative diseases. Neurodegenerative diseases, including Down's syndrome, Alzheimer's disease and Parkinson's disease, are associated with increased levels of certain cytokines, including interleukin-1β (IL-1β) (see Griffin et al. (1989); Mogi et al. (1996)). It has also been shown that IL-1β inhibits long-term potentiation in the hippocampus (Murray, C. A. et al. (1998)). Long-term potentiation in the hippocampus is a form of synaptic plasticity and is generally considered to be an appropriate model for memory and learning (Bliss, T. V. P. et al. (1993)). Thus, inappropriate cytokine expression in the brain is currently believed to be involved in the development and progression of neurodegenerative diseases.

Thus, the invention is indicated for the treatment and prophylaxis of a wide variety of mammalian neuroinflammatory, neurodegenerative and other neurological disorders, including Downs syndrome, Alzheimer's disease, Parkinson's disease, senile dementia, depression, Huntingdon's disease, peripheral neuropathies, Guillain Barr syndrome, spinal cord diseases, neuropathic joint diseases, chronic inflammatory demyelinating disease, neuropathies including mononeuropathy, polyneuropathy, symmetrical distal sensory neuropathy, neuromuscular junction disorders, myasthenias and amyotrophic lateral sclerosis (ALS). Treatment and prophylaxis of these neurodegenerative diseases represents a particularly preferred embodiment of the invention, with treatment of Alzheimer's, ALS and Parkinson's disease particularly preferred.

Regarding disorders involving endothelial dysfunction, the present invention is indicated for the treatment and prophylaxis of a wide variety of such mammalian disorders including, but not limited to, cardiovascular diseases, such as atherosclerosis, peripheral arterial or arterial occlusive disease, congestive heart failure, cerebrovascular disease (stroke), myocardial infarction, angina, hypertension, etc., vasospastic disorders such as Raynaud's disease, cardiac syndrome X, migraine etc., and the damage resulting from ischemia (ischemic injury or ischemia-reperfusion injury). In summary, it can be substantially any disorder the pathology of which involves an inappropriately functioning endothelium.

Further indications for the compositions and processes of the present invention include the treatment of patients to accelerate their rate of wound healing and ulcer healing, and treatment of patients prior to surgical operations, to accelerate their rate of recovery from surgery including their rate of healing of surgical wounds and incisions.

In regard to “cardiac disorders,” the present invention is indicated for the treatment and prophylaxis of a wide variety of such mammalian disorders including, any and all disorders relating to the heart and include, for example, ventricular arrhythmias (ventricular tachycardia or fibrillation) and sudden death from heart disease. Susceptibility of patients to cardiac disorders such as arrhythmias and sudden cardiac death is often indicated by prolonged QT-c intervals in the heart beat rhythm. Administration of compositions according to the preferred embodiments of the invention is believed to reduce QT-c intervals in mammalian patients, indicative of reduced susceptibility to arrhythmia and sudden cardiac death.

The invention is further described, for illustrative purposes, in the following non-limiting examples.

### EXAMPLES

In the examples below, the following abbreviations have the following meanings. If an abbreviation is not defined, it has its generally acceptable meaning.

μl=microliter

μM=micromolar

CHS=contact hypersensitivity

DNFB=2,4-dinitrofluorobenzene

DHS=delayed-type hypersensitivity

EtOH=ethanol

g=gram

hr=hour

IM=intramuscular

kg=kilogram

LPS=lipopolysaccharide

mg=milligram

ml=milliliter

mM=millimolar

ms=millisecond

ng=nanogram

nm=nanometer

nM=nanomolar

PBS=phosphate buffered saline

pg=picagram

### Example 1

Liposomes of size 100±20 nm in average diameter were prepared according to standard methods known in the art and had the following compositions:

Group A—100% POPS (1-palmitoyl-2-oleoyl-sn-glycero-3-[phospho-L-serine], referred to in the examples unless otherwise stated as phosphatidylserine or PS)

Group B—100% POPA (1-palmitoyl-2-oleoyl-sn-glycero-3-phosphate, referred to in the examples unless otherwise stated as phosphatidic acid or PA)

Group C—control, no liposomes

A stock suspension of each liposome composition containing 4.8×10¹⁴ liposomes per ml was diluted with PBS to give an injection suspension containing 6×10⁶ liposomes per ml. The liposomal suspensions were injected into female BALB/c mice (Jackson Laboratories) aged 6-8 weeks and weighing 19-23 g, to determine the effect on ear swelling in the murine CHS model. The CHS model tests for Th1-mediated inflammatory reactions.

The animals were assigned to one of 3 groups, with 5 animals in each group. Groups A and B received approximately 3×10⁵ of the above-identified liposomes (i.e. 100% PS and 100% PA respectively), in a volume of approximately 50 μl. Group C was a control group, receiving no liposomes.

Protocol

The following experiments were performed:

TABLE 1

| | | | | | | | | Day 7 |
|---|---|---|---|---|---|---|---|---|
| Group | Liposomes | Day 1 | Day 2 | Day 3 | Day 4 | Day 5 | Day 6 | (24 hours) |
| A | 100% | Injected then | Injected | Injected | Injected | Injected | Injected then | Ear |
| | PS | sensitized | | | | | challenged | measured |
| B | 100% | Injected then | Injected | Injected | Injected | Injected | Injected then | Ear |
| | PA | sensitized | | | | | challenged | measured |

On days 1-6, mice of Groups A and B were injected with the respective liposomes. Liposomes were injected in 50 μl volumes via intramuscular (IM) injection, i.e. 300,000 liposomes per injection, for a total administration over the test period of 1,800,000 liposomes. Mice of the control group (Group C) received no liposomes, but were sensitized, challenged and tested in the same way as the other groups of mice, as described below.

Sensitization

On day 1, following liposome injection for that day, mice were anaesthetized using 0.2 ml intraperitoneal (IP) injection of 5 mg/ml pentobarbital sodium. The abdominal skin of the mouse was sprayed with 70% EtOH. A blade was used to remove about a one-inch diameter of hair from the abdomen. The bare area was painted with 25 μl of 0.5% 2,4-dinitrofluorobenzene (DNFB) in 4:1 acetone:olive oil using a pipette tip.

Challenge

On day 6, following liposome injection for that day, mice were challenged with DNFB as follows: 10 μl of 0.2% DNFB was painted on the dorsal surface of the right ear with a pipette tip and 10 μl of vehicle was painted on the left ear with a pipette tip.

Results

On day 7, 24 hours after challenge, each animal was anaesthetized with halothane, and ear thickness was measured (in μm) using a Peacock spring-loaded micrometer. Increase in ear swelling was used as a measure of CHS response. Data was expressed as the difference in the treated right ear thickness minus the thickness of the vehicle treated left ear. The experiments were repeated three times, on different but similar animals. The significance of difference between the various groups was determined by the two-tailed student's t-test. A value of p<0.05 was considered significant.

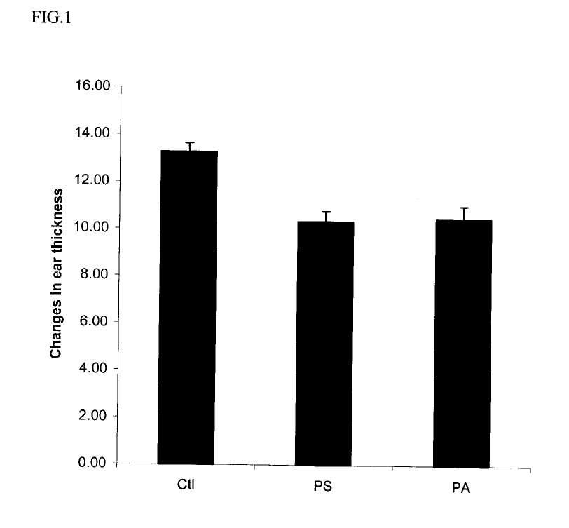

The results are presented graphically in FIG. 1, a bar graph of ear swelling, in ρm. The mean value from the respective experiments was used in compiling the graph.

FIG. 1 shows that a significant reduction in ear swelling (×10⁻² mm) is achieved by injection of liposomes according to the present invention. The reduction achieved with 100% PA liposomes is substantially equivalent to that from 100% PS liposomes, previously reported as anti-inflammatory.

### Example 2

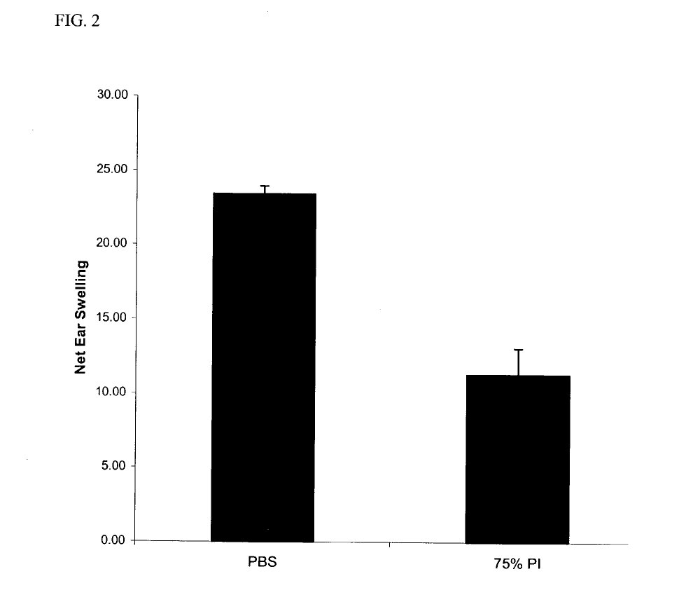

Liposomes consisting essentially of 75% L-α-phosphatidylinositol (referred to in the examples unless otherwise stated as phosphatidylinositol or PI) and 25% 1-palmitoyl-2-oleoyl-sn-glycero-3-phosphocholine or POPC (referred to in the examples unless otherwise stated as phosphatidylcholine or PC); were prepared and tested in the mouse CHS model described above. Two groups of ten mice were sensitized on day 1. One group was injected, on days 1, 2, 3, 4, 5 and 6 with 75% PI: 25% PC liposomes, 600,000 vesicles in 50 μL suspension for each injection. On day 6 after the liposome injection, the mice were challenged with DNFB painted on the left ear as described in Example 1. The second control group was administered 50 μL of PBS according to the same schedule, and similarly challenged. Ear thickness measurements were made on day 7. The results, in bar graph form, are presented in FIG. 2. The data represented the mean ear thickness (×10⁻² mm) changes +/− standard error of the mean (SEM). There was a significant suppression of CHS in the PI liposome injected mice, compared with the PBS control group (p<0.01).

### Example 3

Liposomes of formulation 75% PI and 25% PC and 100±20 nm in average diameter were compared according to standard methods. Five groups (A-E) of 10 mice were sensitized, injected and challenged in accordance with the procedure and schedule described in Example 1. The following numbers of liposomes were delivered in a 50 μl suspension:

Group A 6×10¹⁰

Group B 6×10⁸

Group C 6×10⁷

Group D 6×10⁶

Group E 6×10⁵

Group F 6×10⁴

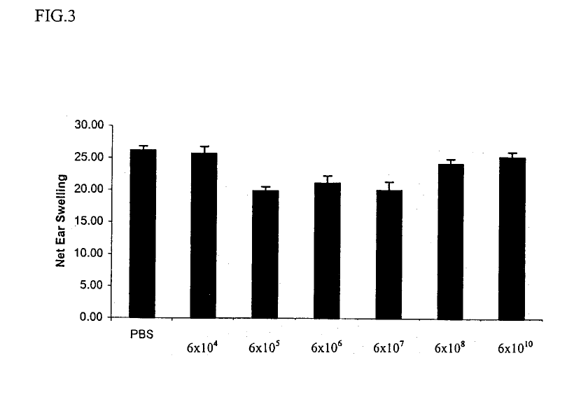

The results, along with a PBS control group, are presented as Net Ear Swelling (×10⁻² mm)+/− SEM in bar graph form in FIG. 3. A significant dose dependent reduction in ear swelling, as compared with the control group, was shown for Group C (6×10⁷), where p=0.05, for Group D (6×10⁶), where p=0.01 and most significantly for Group E where p<0.001. There were no significant differences between the other groups and the control. (Statistical significance calculated by paired student's t-test).

### Example 4

A stock suspension of 75% PI liposomes of size 100±20 nm containing 4.8×10¹⁴ liposomes per ml was diluted to give an injection suspension containing 6×10⁶ liposomes per ml. The liposomal suspensions were used to inject into mice, to determine the effect on ear swelling in the murine DHS model. As in Example 1, female BALB/c mice (Jackson Laboratories) aged 6-8 weeks and weighing 19-23 g were used.

The animals were assigned to one of 2 groups, 10 animals in each group. One group received the liposome injections. The other was a control group that received PBS injections. Each test animal was injected with 50 μl of suspension containing 6×10⁵ liposomes.

Protocol

Mice were sensitized on day 1, challenged on day 6, challenged a second time on day 12, and injected on days 13, 14, 15, 16, 17 and 18 with the 75% PI liposomes as indicated below. On day 18, after the liposome injections, the mice were challenged. Liposomes were injected in 50 μl volume via IM injection, i.e. 600,000 liposomes per injection, for a total administration over the test period of 3,600,000 liposomes. Sensitization and challenge took place as described in Example 1. Ear thickness was measured on day 19.

Results

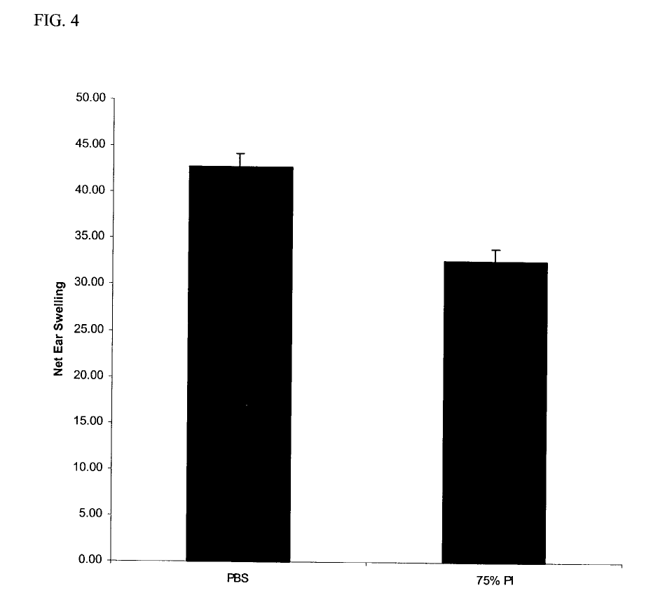

The results are presented in bar graph form on accompanying FIG. 4. The results are expressed as net ear swelling (×10⁻² mm). They show that 75% PI liposomes are effective in the DHS model on day 19, 24 hours after the third injection following the third challenge

### Example 5

U937 is a monocytic leukemia cell line that can be differentiated into macrophages by administration of a phorbol ester. Treatment with LPS, a component of the cell wall of Gram-negative bacteria, stimulates an inflammatory response in U937 cells, with the upregulation of expression of a number of inflammatory molecules including TNFα. This provides an experimental system for the assessment of anti-inflammatory therapies. The macrophages can be grown in culture medium in the presence of a suspected anti-inflammatory composition, and the expression of TNFα measured.

Liposomes of size 100±20 nm were prepared according to standard methods known in the art and had a composition of 75% phosphatidylinositol (PI), 25% phosphatidylcholine (PC). The stock concentration of liposome was 39.5 mM lipid and was diluted to the following final concentrations in the assay:

100 μM phosphatidylinositol (PI)

39.8 μM PI

10 μM PI

3.98 μM PI

1 μM PI

The U937 cells were cultured by growing in RPMI medium (GIBCO BRL) with 10% fetal calf serum (FCS) and 1% penicillin/streptomycin at 37° C., in an atmosphere containing 5% CO₂. They were seeded into 6 well plates at a concentration of 5×10⁵ cells per ml with 2 mls of cells added per well and differentiated into macrophages by treating with 150 nM phorbol myristate acetate (PMA) for 2-3 days. The cell medium was then replaced with complete medium after the U937 cells had differentiated into macrophages. The cells were incubated for an additional 24 hrs prior to liposome addition, so as to allow any up-regulation of genes/proteins induced by PMA to be reduced.

The cells were then incubated with either:

Phosphate buffered saline (PBS)—as a negative control,

10 ng/ml Lipopolysaccharide (LPS)—as a positive control,

10 ng/ml LPS+100 μM PI,

10 ng/ml LPS+39.8 μM PI,

10 ng/ml LPS+10 μM PI,

10 ng/ml LPS+3.98 μM PI,

or 10 ng/ml LPS+1 μM PI.

The cells were incubated at 37° C., 5% CO₂. After 18 hrs, the supernatants from each treatment were collected and assayed for TNF-α using a standard Quantikine Enzyme-linked Immunosorbant Assay (ELISA) kit (R&D systems, Minneapolis, USA).

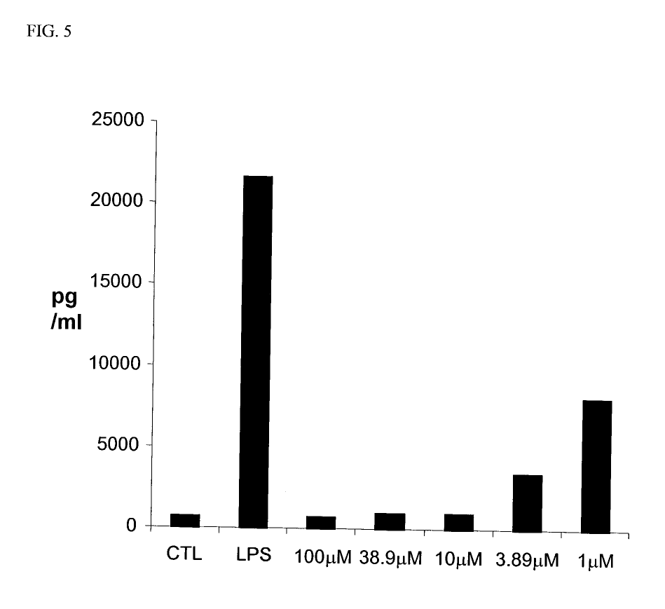

FIG. 5 shows the amount of secreted TNF-α in picagram per ml. The results demonstrate that U937-differentiated macrophage cells express very low levels of TNF-α under normal conditions. However, once exposed to LPS, they secrete large amounts of TNF-α into the surrounding medium, which is indicative of cellular stress occurring. Incubation with the PI liposomes dose dependently decreases the LPS-induced expression of TNF-α.

### Example 6

Sphingolipid metabolism has proved to be a dynamic process, and sphingolipid metabolites—including ceramide, sphingo sine, and sphingosine-1-phosphate—are now recognized as messengers playing essential roles in cell growth, survival and death (Kolesnick, J. Clin. Invest. 110:3-8(2002)). Liposomes consisting essentially of 75% sphingomyelin and 25% PC are prepared according to standard methods (see Katragadda, et al., Cellular and Molecular Biology Letters 5: 483-493 (2000)) and tested in the CHS murine model. The methodology used was as in Example 1 and the size corresponded to those liposomes used in previous examples i.e. 100±20 nm.

What is claimed is:

1. A composition of matter comprising a bodies having a plurality of reactive chemical groups which are capable of interacting with receptors on cells of the mammalian immune system to alter the cytokine profile of the immune system in favor of anti-inflammation, said bodies having a size of about 20-1000 nm, with the proviso that when said groups are all of the same type, said groups are not phosphate-serine or phosphate-glycerol.

2. The composition of claim 1 wherein the bodies comprise reactive chemical groups selected from the group consisting of phosphate-inositol, phosphate-ethanolamine, phosphatidic acid, lysophosphatidic acid, lysophosphate-inositol, lysophosphate-ethanolamine, sphingosine-1-phosphate, ceramides, sphingomyelin, or combinations thereof.

3. The composition according to claim 2, wherein said reactive chemical groups are phosphate-inositol groups.

4. The composition according to claim 2, wherein said bodies are liposomes.

5. The composition according to claim 4, wherein said composition is essentially free of non-lipid pharmaceutically acceptable entities.

6. The composition according to claim 5, wherein said composition is free of non-lipid pharmaceutically acceptable entities.

7. The composition according to claim 5 or 6, wherein the liposomes comprise a phospholipid or glycophospholipid selected from the group consisting of phosphatidylinositol, phosphatidylcholine, phosphatidylethanolamine, phosphatidic acid, distearoylphosphatidylcholine, dipalmitoylphosphatidylcholine, lysophosphatidic acid, lysophosphatidylinositol, lysophosphatidylcholine, lysophosphatidylethanolamine, sphingosylphosphoryl choline, sphingosine-1-phosphate, ceramides, sphingomyelin, or combinations thereof.

8. The composition according to claim 7, wherein the liposome comprises phosphatidylinositol.

9. The composition according to claim 8, wherein the liposomes comprise from about 60 to 100% of phosphatidylinositol.

10. The composition according to claim 9, wherein the liposomes comprise from about 60-90% of phosphatidylinositol.

11. The composition according to claim 9 or claim 10, wherein the remainder of the liposome is phosphatidylcholine.

12. The composition according to claim 11, wherein the liposomes have a size from about 80 nm to about 120 nm.

13. The composition according to any of claims 4-12, wherein said composition comprises from about 10,000 to 2,500,000,000 of said liposomes per unit of administration.

14. A composition of matter capable of producing an anti-inflammatory response in vivo in a mammal, said composition comprising pharmaceutically acceptable bodies of size from about 20 nanometers (nm) to 500 micrometers (μm), comprising a plurality of reactive chemical groups, which interact with receptors on cells of the mammalian immune system to alter the cytokine profile of the immune system in favor of anti-inflammation, with the proviso that said reactive chemical groups are all of the same type, said groups are not phosphate-serine or phosphate-glycerol.

15. The composition according to claim 14, wherein said bodies comprise reactive chemical groups are selected from the group consisting of phosphate-inositol, phosphate-ethanolamine, phosphatidic acid, lysophosphatidic acid, lysophosphate-insitol, lysophosphate-ethanolamine, sphingosine-1-phosphate, ceramides, sphingomyelin, or combinations thereof.

16. The composition according to claim 15, wherein said chemical reactive groups are phosphate-inositol groups.

17. The composition according to claim 14, wherein said bodies are liposomes have a size from about 20-1000 nm.

18. The composition according to claim 17, wherein the composition is essentially free of non-lipid pharmaceutically acceptable entities.

19. The composition according to claim 18, wherein said liposomes comprise a phospholipid or glycophospholipid selected from the group consisting of phosphatidylinositol, phosphatidylcholine, phosphatidylethanolamine, phosphatidic acid, distearoylphosphatidylcholine, dipalmitoylphosphatidylcholine, lysophosphatidic acid, lysophosphatidylinositol, lysophosphatidylcholine, lysophosphatidylethanolamine, sphingosylphosphoryl choline, sphingosine-1-phosphate, ceramides, sphingomyelin, or combinations thereof.

20. The composition according to claim 19, wherein said liposome comprises phosphatidylinositol.

21. The composition according to claim 20, wherein said liposome comprises from about 60 to about 100% phosphatidylinositol.

22. The composition according to claim 21, wherein said liposome comprises from about 60 to about 90% phosphatidylinositol.

23. The composition according to claim 21, wherein the remainder of the liposome is phosphatidylcholine.

24. The composition according to claim 22, wherein the remainder of the liposome is phosphatidylcholine.

25. The composition according to claim 17, wherein the composition is free of non-lipid pharmaceutically acceptable entities.

26. The composition according to claim 25, wherein said liposomes comprise a phospholipid or glycophospholipid selected from the group consisting of phosphatidylinositol, phosphatidylcholine, phosphatidylethanolamine, phosphatidic acid, distearoylphosphatidylcholine, dipalmitoylphosphatidylcholine, lysophosphatidic acid, lysophosphatidylinositol, lysophosphatidylcholine, lysophosphatidylethanolamine, sphingosylphosphoryl choline, sphingosine-1-phosphate, ceramides, sphingomyelin, or combinations thereof.

27. The composition according to claim 26, wherein said liposomes comprise phosphatidylinositol.

28. The composition according to claim 27, wherein said liposomes comprise from about 60 to about 100% phosphatidylinositol.

29. The composition according to claim 28, wherein said liposomes comprise from about 60 to about 90% phosphatidylinositol.

30. The composition according to claim 28, wherein the remainder of the liposomes are phosphatidylcholine.

31. The composition according to claim 29, wherein the remainder of the liposomes are phosphatidylcholine.

32. The composition as in any of claims 14-30 or 31, wherein said composition comprises from about 10,000 to about 2,500,000,000 of liposomes per unit of administration.

33. A method for treating a T-cell function-mediated disorder comprising administering to a mammalian patient an effective amount of pharmaceutically acceptable bodies comprising an effective number of anti-inflammatory promoting groups to inhibit and/or reduce the progression of the T-cell function-mediated disorder.

34. The method according to claim 33, wherein said bodies are liposomes.

35. The method according to claim 34, wherein said liposomes have a size from about 20 to about 1000 nm.

36. The method according to claim 35, wherein said liposomes comprise from about 60 to about 100% phosphatidylinositol.

37. The method according to claim 36, wherein said liposomes comprise from about 60 to about 90% phosphatidylinositol.

38. A method for treating an inflammatory disorder comprising administering to a patient an effective amount of pharmaceutically acceptable bodies comprising an effective number of anti-inflammatory promoting groups, to inhibit and/or reduce the progression of the inflammatory disorder.

39. The method according to claim 38, wherein said bodies are liposomes.

40. The method according to claim 39, wherein said liposomes have a size from about 20 to about 1000 nm.

41. The method according to claim 40, wherein said liposomes comprise from about 60 to about 100% phosphatidylinositol.

42. The method according to claim 41, wherein said liposomes comprise from about 60 to about 90% phosphatidylinositol.

43. A method for treating an endothelial function disorder comprising administering to a mammalian patient an effective amount of pharmaceutically acceptable bodies comprising an effective number of anti-inflammatory promoting groups, to inhibit and/or reduce the progression of the endothelial function disorder.

44. The method according to claim 43, wherein said bodies are liposomes.

45. The method according to claim 44, wherein said liposomes have a size from about 20 to about 1000 nm.

46. The method according to claim 45, wherein said liposomes comprise from about 60 to about 100% phosphatidylinositol.

47. The method according to claim 46, wherein said liposomes comprise from about 60 to about 90% phosphatidylinositol.

48. A method for treating an immune disorder characterized by inappropriate cytokine expression comprising administering to a mammalian patient an effective amount of pharmaceutically acceptable bodies comprising an effective number of anti-inflammatory promoting groups, to inhibit and/or reduce the progression of the immune disorder.

49. The method according to claim 48, wherein said bodies are liposomes.

50. The method according to claim 49, wherein said liposomes have a size from about 20 to about 1000 nm.

51. The method according to claim 50, wherein said liposomes comprise from about 60 to about 100% phosphatidylinositol.

52. The method according to claim 51, wherein said liposomes comprise from about 60 to about 90% phosphatidylinositol.

53. A method for treating or prophylaxis of a mammalian cardiac disorder, the presence of or the susceptibility to which is detectable by observing a prolonged QT-c interval on an electrocardiogram of the patient, which method comprises administering to the patient suffering therefrom or susceptible thereto a pharmaceutical composition comprising pharmaceutically acceptable bodies, and a pharmaceutically acceptable carrier, wherein at least a portion of said bodies are in the range from about 20 nm to 500 μm, and wherein the surfaces of said bodies have been modified to comprise, as a major component, at least one anti-inflammatory promoting group, with the proviso that said anti-inflammatory group is not phosphate serine phosphate-glycerol and phosphate-serine.

54. The method according to claim 53, wherein said bodies are liposomes.

55. The method according to claim 54, wherein said liposomes have a size from about 20 to about 1000 nm.

56. The method according to claim 55, wherein said liposomes comprise from about 60 to about 100% phosphatidylinositol.

57. The method according to claim 56, wherein said liposomes comprise from about 60 to about 90% phosphatidylinositol.

58. A method for treating a T-cell function-mediated disorder comprising administering to a mammalian patient suffering from or at risk of suffering from a T-cell function-mediated disorder, an effective amount of a composition comprising pharmaceutically acceptable bodies having a size from about 20 nm to about 500 μm, comprising on the surface thereof a plurality of anti-inflammatory promoting groups other than phosphate-glycerol or phosphate-serine, or groups convertible to said anti-inflammatory promoting groups, such that upon administration, the progression of the T-cell function mediated disorder is inhibited and/or reduced.

59. The method according to claim 58, wherein said bodies are liposomes.

60. The method according to claim 59, wherein said liposomes have a size from about 20 to about 1000 nm.

61. The method according to claim 60, wherein said liposomes comprise from about 60 to about 100% phosphatidylinositol.

62. The method according to claim 61, wherein said liposomes comprise from about 60 to about 90% phosphatidylinositol.

63. A method for treating an endothelial function disorder comprising administering to a mammalian patient suffering from or at risk of suffering from an endothelial function disorder an effective amount of a composition comprising pharmaceutically acceptable bodies having a size from about 20 nm to about 500 μm, comprising on the surface thereof a plurality of anti-inflammatory promoting groups other than phosphate-glycerol or phosphate-serine, or groups convertible to said anti-inflammatory promoting groups, such that upon administration, the progression of the endothelial function mediated disorder is inhibited and/or reduced.

64. The method according to claim 63, wherein said bodies are liposomes.

65. The method according to claim 64, wherein said liposomes have a size from about 20 to about 1000 nm.

66. The method according to claim 65, wherein said liposomes comprise from about 60 to about 100% phosphatidylinositol.

67. The method according to claim 66, wherein said liposomes comprise from about 60 to about 90% phosphatidylinositol.

68. A method for treating an immune disorder comprising administering to a mammalian patient suffering from or at risk of suffering from an immune disorder an effective amount of a composition comprising pharmaceutically acceptable bodies having a size from about 20 nm to about 500 μm, comprising on the surface thereof a plurality of anti-inflammatory promoting groups other than phosphate-glycerol or phosphate-serine, or groups convertible to said anti-inflammatory promoting groups, such that upon administration, the progression of the immune disorder is inhibited and/or reduced.

69. The method according to claim 68, wherein said bodies are liposomes.

70. The method according to claim 69, wherein said liposomes have a size from about 20 to about 1000 nm.

71. The method according to claim 70, wherein said liposomes comprise from about 60 to about 100% phosphatidylinositol.

72. The method according to claim 71, wherein said liposomes comprise from about 60 to about 90% phosphatidylinositol.

73. A method for treating an inflammatory disorder comprising administering to a mammalian patient suffering from or at risk of suffering from an inflammatory disorder an effective amount of a composition comprising pharmaceutically acceptable bodies having a size from about 20 nm to about 500 μm, comprising on the surface thereof a plurality of anti-inflammatory promoting groups, other than phosphate-glycerol or phosphate-serine, or groups convertible to said anti-inflammatory promoting groups, such that upon administration, the progression of the inflammatory disorder is inhibited and/or reduced.

74. The method according to claim 73, wherein said bodies are liposomes.

75. The method according to claim 74, wherein said liposomes have a size from about 20 to about 1000 nm.

76. The method according to claim 75, wherein said liposomes comprise from about 60 to about 100% phosphatidylinositol.

77. The method according to claim 76, wherein said liposomes comprise from about 60 to about 90% phosphatidylinositol.

78. The method as in claims 33-77, wherein the amount said bodies administered is less than 30 mg per kg patient body weight.

79. The method as in claims 33-77, wherein the number of said bodies administered is from about 500 to about 2.5×10⁹ bodies.

80. The method as in claims 33-77, wherein said bodies are essentially free of non-lipid pharmaceutically acceptable entities.

81. The method as in claims 33-77, wherein said bodies are free of non-lipid pharmaceutically acceptable entities.

82. The method as in any of claims 36, 41, 46, 51, 56, 61, 66, 71, or 76, wherein remainder of said liposome is phosphatidylcholine.

83. The method as in any of claims 37, 42, 47, 52, 57, 62, 67, 72, or 77, wherein remainder of said liposome is phosphatidylcholine.

84. A pharmaceutical composition in unit dosage form, for administration to a mammalian patient, comprising pharmaceutically acceptable bodies and a pharmaceutically acceptable carrier, wherein at least a portion of the bodies have a size in the range from about 20 nm to 500 μm, and wherein the surfaces of said bodies comprise anti-inflammatory promoting groups, said unit dosage comprising from 500 to 2.5×10⁹ bodies, wherein said group is an anti-inflammatory promoting group other than phosphate-glycerol and phosphate-serine.

85. The pharmaceutical composition according to claim 84, wherein said bodies are liposomes.

86. The pharmaceutical composition according to claim 85, wherein said liposomes have a size from about 20 to about 1000 nm.

87. The pharmaceutical composition according to claim 86, wherein said composition is essentially free of non-lipid pharmaceutically acceptable entities.

88. The pharmaceutical composition according to claim 87, wherein said composition is free of non-lipid pharmaceutically acceptable entities.

89. The pharmaceutical composition according to claim 87, wherein the liposome comprise from about 60-100% of phosphatidylinositol.

90. The pharmaceutical composition according to claim 88, wherein the liposome comprise from about 60-100% of phosphatidylinositol.

91. The pharmaceutical composition according to claim 89, wherein the liposome comprise from about 60-90% of phosphatidylinositol.

92. The pharmaceutical composition according to claim 90, wherein the liposome comprise from about 60-90% of phosphatidylinositol.

93. The pharmaceutical composition as in one of claims 89-92, wherein the remainder of the liposome comprises phosphatidylcholine.
